(19) United States
(12) Patent Application Publication
(10) Pub. No.: US 2005/0166929 A1
(43) Pub. Date: Aug. 4, 2005

(54) Methods and devices for relieving upper airway obstructions

(75) Inventor: Yandong Jiang, North Reading, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: MASSACHUSETTS GENERAL HOSPITAL, Boston, MA (US)

(21) Appl. No.: 11/021,157

(22) Filed: Dec. 22, 2004

Related U.S. Application Data
(63) Continuation-in-part of application No. 10/769,180, filed on Jan. 30, 2004.

(51) Int. Cl. A61F 5/56; A61C 5/14

(52) U.S. Cl. 128/861; 128/848

(57) ABSTRACT

Methods and devices are provided that are effective to remove an obstruction in a human airway related to snoring and/or OSA. In one embodiment, the device includes a mouthpiece that is adapted to form a sealed cavity within a human mouth, and a hollow elongate member having a first end that is coupled to the mouthpiece and that is in communication with the sealed cavity, and a second end that is adapted to be coupled to a negative pressure generator. In use, a negative pressure generator can be attached to the hollow elongate member to create a negative pressure in a human mouth in response to an obstructed airway, thereby removing the obstruction. In particular, this device is effective to pull a patient's tongue and/or soft tissues of the upper airway up and away from the posterior pharyngeal wall to reopen the airway.

### CROSS-REFERENCE TO RELATED APPLICATIONS

This application is a continuation-in-part of U.S. patent application Ser. No. 10/769,180 filed on Jan. 30, 2004 and entitled “Methods and Devices for Relieving Upper Airway Obstructions,” which is hereby incorporated by reference in its entirety.

### FIELD OF THE INVENTION

The present invention relates to methods and devices for reducing snoring and/or airway obstructive events, and in particular to methods and devices that are effective to generate a negative pressure in a patient's mouth to remove an upper airway obstruction and reduce or eliminate snoring and/or airway obstruction.

### BACKGROUND OF THE INVENTION

Over 60 million Americans are affected by snoring and/or obstructive sleep apnea (OSA). During normal waking hours, muscle tone in most individuals unconsciously maintains the tongue, pharyngeal folds, soft palate, uvula, epiglottis and posterior pharyngeal wall in adequate spatial relationships so as not to interfere with the free passage of air. However, when asleep in the supine position, gravity can cause the tongue, soft palate, uvula, and epiglottis to move back toward the posterior pharyngeal wall. As a result, the size of the upper airway can be reduced and snoring may occur. Moreover, snoring may also be a sign that a person is suffering from OSA.

OSA is a condition where a person temporarily stops breathing for a short amount of time (10 seconds or longer) due to the blockage of the airway. During a customary sleep period a person suffering from OSA can experience hundreds of so called apneatic events, that is, periods when the person's airway becomes blocked until the patient's hypoxia becomes severe enough that the person awakens and resumes breathing normally again. Not only do these apneatic events cause a deficiency of restful sleep but, due to depleted oxygen levels, possible long term health problems, such as pulmonary hypertension, heart failure and stroke, can result.

One common non-invasive treatment approach for OSA is the use of a continuous positive airway pressure (CPAP) machine. A CPAP machine uses a nasal mask, harness or other headgear to continuously deliver pressured air directly to the person's windpipe, and the positive pressure prevents the upper airway from collapsing during sleep. While proven effective, most CPAP users often suffer from at least one of the following side effects: claustrophobia, difficulty exhaling, inability to sleep, nasal congestion, sore eyes, sore or dry throat, headaches, abdominal bleeding, chest muscle discomfort, nosebleeds and mask-related problems such as rash, skin abrasions and conjunctivitis from air leakage. Additionally, and especially during the early stages of usage, some people may have difficulty adjusting to both the mechanism and/or sound of the machine.

Alternatives to the CPAP machine include devices which can lock the tongue in a fixed position, such as metallic or hard plastic clips. However, these devices risk pain and injury to the tongue as well as are unsuited for self administration. Another alternative to the CPAP machine are mouthpieces that are effective to create an enlarged airway and/or hold the tongue in a fixed position using some type of retainer. While mouthpieces have had some success, normal swallowing can be interrupted, causing a reduction in the clearance of airway secretion, saliva aspiration, and even gastric reflex. Further, a mouthpiece may also cause temporomandibular joint pain to occur and can be detrimental to the normal bite relationship of the dental arches, since it distorts the relationship of the upper and lower jaws. More recently, some mouthpieces have attempted to use a vacuum to hold the tongue, or a portion thereof, in the retainer. However, these devices are often cumbersome and distracting to the sleeping patient. Further, should the patient swallow, the vacuum is broken and the tongue is pulled out of the retainer, resulting in an airway obstruction and a high degree of patient discomfort.

Accordingly, there remains a need for improved, non-invasive treatment methods and devices that are effective to remove upper airway obstructions, reduce or eliminate snoring and/or apneatic events and the related complications, and improve the patent's sleeping quality.

### SUMMARY OF THE INVENTION

The present invention generally provides methods and devices that are effective to remove an obstruction in a human airway related to snoring and/or OSA. In one embodiment, the device includes a mouthpiece that is adapted to form a sealed cavity within a human mouth without impinging on a tongue in the mouth, and a hollow elongate member having a first end that is coupled to the mouthpiece and that is in communication with the sealed cavity, and a second end that is adapted to be coupled to a negative pressure generator. In use, a negative pressure generator can be attached to the hollow elongate member to remove air from the sealed cavity. When an obstructed airway occurs as a result of the collapse of the soft tissues of the upper airway, a negative pressure is created within the sealed cavity to pull the tongue and other soft tissues of the upper airway away from the posterior pharyngeal wall, thereby removing the obstruction. The “soft tissues of the upper airway” include, but are not limited to, the tongue, pharyngeal folds, soft palate, uvula, epiglottis and posterior pharyngeal wall.

While the mouthpiece can have a variety of configurations, in one embodiment it includes upper and lower portions that are adapted to conform to the anatomy of a human's upper and lower dental structures. The upper and lower portions of the mouthpiece are preferably connected to one another by a sidewall that extends therebetween to form the sealed cavity within the mouth. In another embodiment, the mouthpiece can include a sidewall that is adapted to be positioned over the opening of a human mouth, and a positioning member that is coupled to the sidewall and that is adapted to fit within the mouth to maintain the mouthpiece at a fixed position. The positioning member can include, for example, opposed first and second fixation elements that are adapted to be positioned between the upper and lower dental structures.

The present invention also provides a negative pressure generator for use within a mouthpiece or similar device that formS a sealed cavity within the patient's mouth. While the negative pressure generator can have a variety of configurations, in one embodiment it can be adapted to be operated by a patient's own breathing. In particular, the negative pressure generator can be in the form of a deformable member that is adapted to deform in response to inhalation, and that is biased to an original, un-deformed state such that return to the original, un-deformed state is effective to generate a negative pressure within the sealed cavity when an airway obstruction occurs. The negative pressure generator can also include at least one one-way valve that is adapted to control air flow into and out of the deformable member. A mating element, such as a strap or belt, can be used to releasably mate the deformable member to the patient.

In another embodiment, the present invention provides a method for removing an obstruction in a human airway by forming a sealed cavity within a human mouth and coupling the sealed cavity to a negative pressure generator. When an obstructed airway is caused by the collapse of the soft tissues of the upper airway, the negative pressure generator creates a negative pressure within the sealed cavity of the mouth to pull the tongue and/or soft tissues of the upper airway away from the posterior pharyngeal wall, thereby re-opening the obstructed airway.

### BRIEF DESCRIPTION OF THE DRAWINGS

The invention will be more fully understood from the following detailed description taken in conjunction with the accompanying drawings, in which:

FIG. 1A is a side perspective view of one embodiment of a device for removing an airway obstruction in accordance with the present invention;

FIG. 1B is a front perspective view of the device shown in FIG. 1A;

FIG. 2 is a side view of a device for removing an airway obstruction having an expanded sidewall in accordance with another embodiment of the present invention;

FIG. 3 is a top perspective view of yet another embodiment of a device for removing an airway obstruction having a positioning member in accordance with the present invention;

FIG. 4 is a side perspective view of one embodiment of a negative pressure generator that is coupled to a human chest, and that is operated by the human breathing;

FIG. 5A is a side view illustration of the oral anatomy of a human, showing an obstructed human airway in accordance with the present invention; and

FIG. 5B is a side view illustration of the oral anatomy shown in FIG. 5A, showing the human airway after the obstruction is removed using a negative pressure generator in accordance with the present invention.

### DETAILED DESCRIPTION OF THE INVENTION

The present invention provides a non-invasive remedy for snoring and/or OSA that is effective to generate a negative pressure within the oral cavity of a human to relieve an obstructed airway. In particular, when a human, referred to herein as a patient, is sleeping in the supine position, the soft tissues of the upper airway may fall against the posterior wall of the pharynx, thus blocking the air passageway. In response, the device of the present invention is effective to generate a negative pressure in the patient's mouth that pulls the patient's soft tissues of the upper airway apart to reopen the airway. The device is particularly advantageous in that, when a patient's upper airway is open, the negative pressure generator will remove only a small amount of air from the oral cavity without the creation of a negative pressure in the oral cavity, thus allowing the patient to breathe normally and comfortably. A negative pressure is only created when the airway is obstructed. Accordingly, the device does not impinge on the tongue, allowing free movement of the tongue when the airway is unobstructed, thereby reducing the risk of choking, coughing or aspiration due to excess saliva.

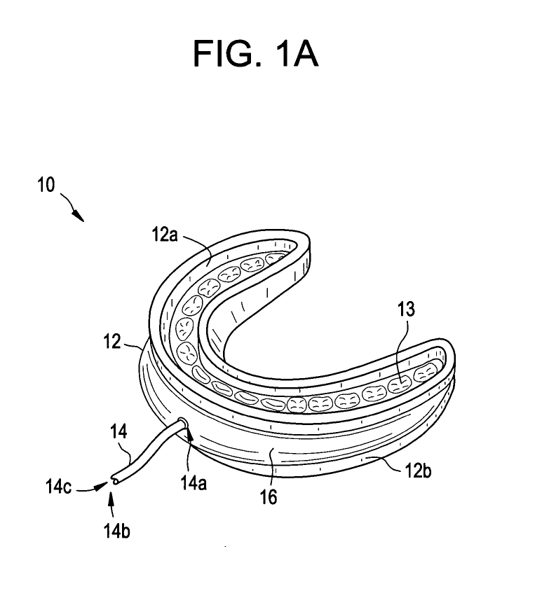

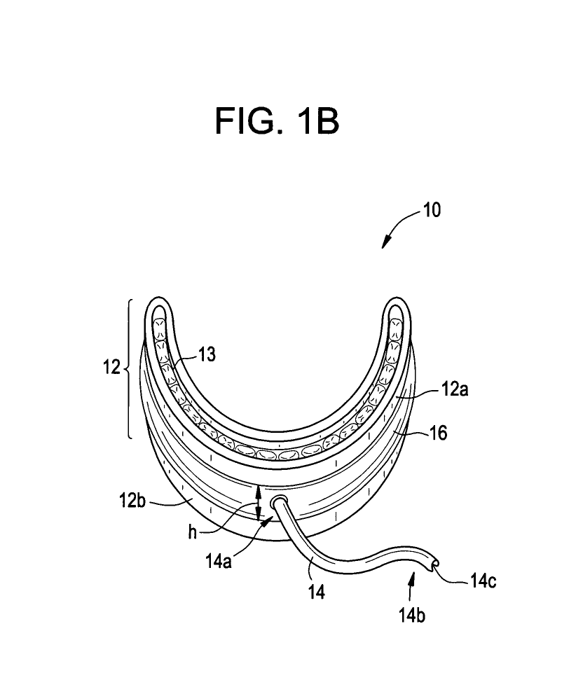

FIGS. 1A and 1B illustrate an exemplary embodiment of a device 10 for removing an airway obstruction in accordance with the present invention. As shown, the device 10 generally includes a mouthpiece 12 that is adapted to fit within a patient's mouth and that is effective to form a sealed cavity within the patient's mouth, and a hollow elongate member 14 having a first end 14a that is coupled to the mouthpiece 12 and that is in communication with the sealed cavity, and a second end 14b that is adapted to couple to a negative pressure generator.

The mouthpiece 12 can have a variety of configurations, but it is preferably effective to create a sealed cavity within the patient's mouth to allow a negative pressure to be created therein in response to an obstructed airway. A sealed cavity can be formed by blocking the passage of air through the patient's mouth, such that the patient is prevented from breathing through the mouth. The sealed cavity should, however, be in communication with the patient's upper airway. This will allow the negative pressure generator to create a negative pressure within the sealed cavity when an obstruction occurs in the patient's upper airway.

In the illustrated embodiment, a sealed cavity is formed using a mouthpiece 12 that includes upper and lower portions 12a, 12b that are sized to fit around and/or to receive some or all of the upper and lower dental structures of the patient, including the gums and/or teeth, and a sidewall 16 that extends between the upper and lower portions 12a, 12b. Each portion 12a, 12b can have a variety of shapes and sizes and they can be individually molded to provide a custom fit, or, alternatively, each portion 12a, 12b can have a universal shape and size for use by most patients. The upper and lower portions 12a, 12b should, however, be effective to facilitate and maintain placement of the mouthpiece 12 in the patient's mouth. As shown in FIGS. 1A-1B, the upper and lower portions 12a, 12b are each substantially U-shaped, and they include cavities (only one cavity 13 is shown in upper portion 12a) formed therein for receiving the patient's teeth and/or gums. When positioned in the patient's mouth, the upper and lower portions 12a, 12b extend around the upper and lower incisors, canine teeth, and some of the pre-molar teeth.

The sidewall 16 that extends between the upper and lower portions 12a, 12b, can also have a variety of configurations, and it can be integrally formed with the upper and lower portions 12a, 12b, or it can be connected to the upper and lower portions 12a, 12b to mate the portions 12a, 12b to one another. The sidewall 16 should, however, extend between the upper and lower portions 12a, 12b such that it is positioned adjacent to the patient's lips when the mouthpiece 12 is in use. This will allow the sidewall 16 to prevent air from entering the patient's mouth, thus allowing a negative pressure to be created in the patient's mouth in response to a blocked airway.

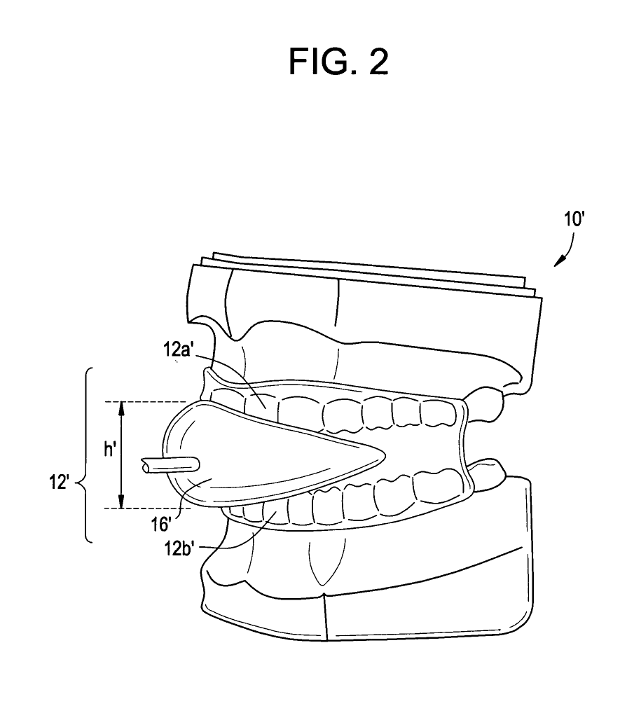

In another embodiment of the present invention, the sidewall 16 can be adapted to expand the size of the oral cavity or sealed cavity in the patient's mouth. An expanded oral cavity allows the tongue and/or soft palate to be pulled a greater distance away from the posterior pharyngeal wall, thus making it more difficult for the tongue and other soft tissues to form a complete blockage of the upper airway when they collapse. This is particularly advantageous for patients having a relatively small oral cavity, a large tongue, and/or patients lacking teeth. While a variety of techniques can be used to expand the size of the oral cavity, in one embodiment the sidewall 16 can be configured to maintain the patient's dental structures in a fixed relationship with respect to one another. In particular, the sidewall 16 can have a height h that extends between the upper and lower portions 12a, 12b of the mouthpiece 12 such that the patient's dental structures are positioned a distance apart from one another that is substantially equal to the height h of the sidewall 16, as shown in FIG. 1B. In another embodiment, as shown in FIG. 2, the sidewall 16, can be in the form of a protrusion that extends between the upper and lower portions 12a′, 12b′ and that protrudes through the mouth of the patient such that part of the sidewall 16′ is positioned between the patient's lips. The protruding sidewall 16′ can vary in shape and size, but in an exemplary embodiment it is substantially rounded and it has a width (not shown) that allows the sidewall 16′ to extend between opposed edges of a patient's lips, and a height h′ that is effective to maintain the patient's upper and lower dental structures at a fixed distance apart from one another. A person skilled in the art will appreciate that the mouthpiece can have a variety of other configurations to expand the size of a patient's oral cavity.

For example, in another embodiment (not shown), the sidewall 16 can extend at an angle from top portion 12a, 12a′ to lower portion 12b, 12b′ such that the lower portion 12b, 12b′ holds the lower jaw in a forward position relative to its resting state, so as to expand the size of the oral cavity or sealed cavity in the patient's mouth. One skilled in the art will appreciate that the extent by which the lower portion may be advanced or displaced with respect to the upper portion will vary depending on the needs and anatomy of individual patients. In one aspect the lower portion may be advanced with respect to the upper portion by an amount in the range of about 0.25 mm to 30 mm, more preferably in the range from about 2 mm to 14 mm, and most preferably in the range from about 4 mm to 6 mm. A person skilled in the art will also appreciate that the mouthpiece can be configured to adjust any amount of advancement of the lower portion with respect to the upper portion by any suitable amount, for example within the range of about 0 to 30 mm. A variety of mechanisms can be used to allow relative movement between the upper and lower portions, and to lock the lower portion in a desired position relative to the upper portion. Such devices include, but are not limited to, screws, slides, pins, rubber bands, etc.

Referring back to FIGS. 1A and 1B, the device 10 also includes a hollow elongate member 14 that is coupled to the mouthpiece 12 and that is in communication with the sealed cavity. The hollow elongate member 14 can have a variety of shapes and sizes, but it should be effective to provide a passageway between the sealed cavity in the patient's mouth and a negative pressure generator. In an exemplary embodiment, the hollow elongate member 14 has a generally tubular shape and it includes a first end 14a that is mated to the mouthpiece 12, and a second end 14b that is adapted to couple to a negative pressure generator. The first end 14a can be removably or fixedly attached to any portion of the mouthpiece 12, but in an exemplary embodiment it is fixedly attached to the sidewall 16 of the mouthpiece 12, preferably at a substantial midpoint thereof, as shown in FIGS. 1A-1B. The length and flexibility of the hollow elongate member 14 can also vary, but it should have a length and flexibility that does not restrict movement of the patient during use. The length should also be sufficient to allow a negative pressure generator, which is attached to the second end 14b of the hollow elongate member 14, to be positioned a distance apart from the patient.

The hollow elongate member 14 also includes an inner lumen 14c extending therethrough between the first and second ends 14a, 14b. The inner lumen 14c is in communication with the sealed cavity in the patient's mouth and the negative pressure generator, thus allowing the negative pressure generator to create a negative pressure within the sealed cavity. The inner lumen 14c can vary in shape and size, but the size should be adapted to allow a negative pressure generator to remove air from the patient's mouth at a rate that is effective to create a negative pressure in the sealed cavity only when an airway obstruction occurs, and to otherwise allow normal breathing and swallowing by the patient. A person skilled in the art will appreciate that the hollow elongate member 14 can have a variety of other configurations, and moreover that a variety of techniques can be used to couple the sealed cavity to a negative pressure generator.

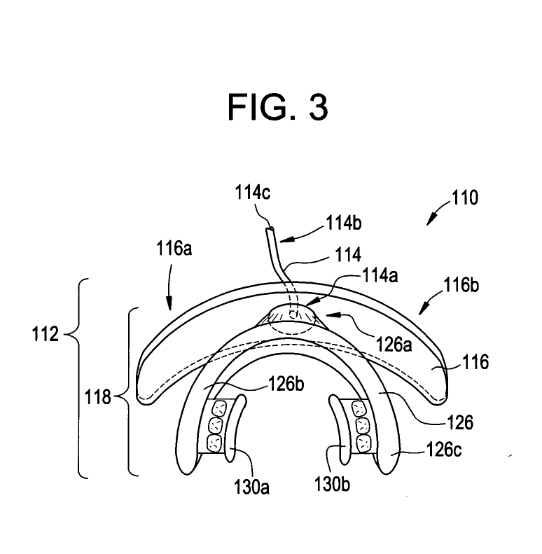

By way of non-limiting example, FIG. 3 illustrates yet another embodiment of a device 110 for removing an airway obstruction. In this embodiment, the device 110 includes a mouthpiece 112 having a sidewall 116 that is adapted to be positioned over the opening of the patient's mouth, rather than within the patient's mouth as described in connection with FIGS. 1A-2. The device 110 also includes a positioning member 118 that is adapted to fit within the patient's mouth to maintain the mouthpiece 112 at a fixed position. A hollow elongate member 114 is coupled to the mouthpiece 112 and it is adapted to provide communication between the sealed cavity and a negative pressure generator. In use, the device 110 operates similar to device 10 (described above in connection with FIGS. 1A-2) in that the sidewall 116 of the mouthpiece 112 prevents air from entering the patient's mouth, thus allowing a negative pressure to be created in the patient's mouth in response to a blocked airway.

While the sidewall 116 of the mouthpiece 112 can have a variety of shapes and size, FIG. 3 illustrates a substantially oval, elongate sidewall 116 that conforms to an external portion of a patient's face. More particularly, the sidewall 116 includes opposed ends 116a, 116b that are preferably angled toward one another and that fit around the area on opposed sides of a patient's lips. The sidewall 116 can also optionally include one or more cavities (not shown) formed therein for seating at least a portion of the patient's lips and/or surrounding facial structures. As stated above, the mouthpiece 112 also includes a positioning member 118 that is connected to the sidewall 116, and that is effective to maintain the mouthpiece 112 at a fixed position with respect to the patient's mouth. While the positioning member 118 can have a variety of configurations, in the illustrated embodiment the positioning member 118 includes a connecting wall 126 that is coupled to a substantial mid-portion of the sidewall 116, and that is adapted to extend into the patient's mouth. The connecting wall 126 has a first, substantially cylindrical portion 126a that is mated to the sidewall 116, and a second portion having opposed extension members 126b, 126c. The first portion 126a is configured to fit between the patient's lips, and the extension members 126b, 126c are configured to extend into the patient's mouth such that they are positioned on opposed sides of the patient's upper and lower dental structures. Each extension portion 126b, 126c can optionally include a fixation element 130a, 130b formed thereon and adapted to be positioned between the upper and lower dental structures of the patient. In an exemplary embodiment, the fixation elements 130a, 130b are adapted to conform to the patient's canine and/or molar teeth to allow the patient to bite down on the fixation elements 130a, 130b, thus maintaining the position of the mouthpiece 112 within the patient's mouth. A person skilled in the art will understand that the embodiment illustrated in FIG. 3 can be modified in various ways. For example, the fixation elements 130a, 130b need not be present and/or extension portion 126b, 126c do not need to extend as far back as the opposite sides of the patient's dentures as the device can seal the oral cavity simply by contacting and conforming to the lips and/or oral cavity.

The device 110 also includes a hollow elongate member 114 which provides a passageway between the sealed cavity in the patient's mouth and a negative pressure generator. The hollow elongate member 114, which is similar to hollow elongate member 14 described above in connection with FIGS. 1A-1B, has a first end 114a coupled to the mouthpiece 112 at a substantial mid-portion of the sidewall 116 such that the inner lumen 114c in the hollow elongate member 114 is in communication with the sealed cavity formed within the patient's mouth, and a second end 114b adapted to mate to a negative pressure generator.

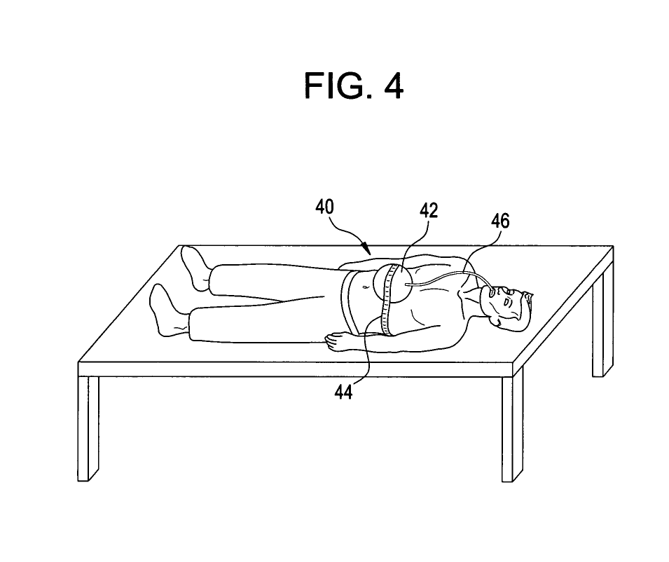

As previously stated, the devices in accordance with the present invention are preferably used in connection with a negative pressure generator that is effective to create and maintain a negative pressure in the sealed cavity in the patient's mouth. While virtually any negative pressure generator that is effective to withdraw air and/or fluid from a sealed cavity can be used, by way of non-limiting example, the negative pressure generator can be a vacuum pump. Alternatively, the negative pressure generator can be a device that is operated using energy generated from the patient's own breathing, and FIG. 4 illustrates an exemplary embodiment of one such device.

As shown in FIG. 4, the negative pressure generator 40 is in the form of a deformable member 42 that is positioned on the patient's chest, and that is preferably held there by a mating element 44. In use, the deformable member 42 is effective to deform when the patient's chest expands due to inhalation, and it is biased to an original, non-deformed state. This will allow the deformable member 42 to draw in air from the sealed cavity in the patient's mouth as the deformable member 42 transitions from a deformed state to its original, non-deformed state, thus creating a negative pressure within the sealed cavity. While the deformable member 42 can have virtually any shape and size, in the illustrated embodiment the deformable member 42 has a generally spherical shape, such that a hollow cavity formed within the deformable member 42 is in communication with a sealed cavity in the patient's mouth.

The deformable member 42 also preferably includes a first one-way valve (not shown) that is adapted to control air flow from the sealed cavity in the patient's mouth, to the inner cavity in the deformable member 42. In particular, the first one-way valve, allows air to be drawn in from the sealed cavity, yet it does not allow air to flow in a reverse direction from the deformable member to the sealed cavity. A second one-way valve is also preferably provided to allow air to be released into the environment as the deformable member 42 deforms, yet to prevent air from being drawn in from the surrounding environment as the deformable member 42 returns to the original, non-deformed state. Accordingly, the first and second one-way valves work in conjunction with one another to control air flow to allow a negative pressure to be created in the sealed cavity in the patient's mouth. In an exemplary embodiment, the first one-way valve is disposed between a hollow elongate member 46 that extends from the sealed cavity to the deformable member 42, and the second one-way valve is disposed at a location on the deformable member 42 that allows air to be released into the surrounding environment.

As previously stated, the deformable member 42 also preferably includes a mating element 44 that is effective to at least temporarily retain the deformable member 42 on the patient's chest during use of the device. While a variety of techniques can be used to couple the deformable member 42 to the patient's chest, FIG. 4 illustrates a strap or belt 44 that is disposed around both the patient's midsection and the deformable member 42, and that is effective to releasably secure the deformable member 42 to the patient's chest. A person skilled in the art will appreciate that virtually any technique can be used to couple the deformable member 42 to the patient's chest including, for example, a jacket that contains the deformable member 42 and that is wearable by the patient.

In use, the deformable member 42 is coupled to the sealed cavity in the patient's mouth, preferably by a hollow elongate member 46 that extends between the deformable member 42 and the sealed cavity, as shown. The hollow elongate member 46 is similar to hollow elongate member 14 described above with respect to FIGS. 1A-1B. When the patient inhales, the deformable member 42 deforms and air is released into the surrounding environment from the second one-way valve. As the patient exhales, the deformable member 42 preferably simultaneously returns to the original, non-deformed state, receiving air from the patient through the hollow elongate member 46 and the first one-way valve. When a blockage occurs due, for example, to the soft tissues of the upper airway falling against the posterior pharyngeal wall, however, air removed from the sealed cavity within the patient's mouth will create a negative pressure, thereby allowing the tongue and/or other soft tissues of the upper airway to be pulled away posterior pharyngeal wall, thus removing the obstruction.

A person skilled in the art will appreciate that the negative pressure generator can have a variety of other configurations, and that a variety of other techniques can be used to create a negative pressure within a sealed cavity in a patient's mouth in response to a blocked airway.

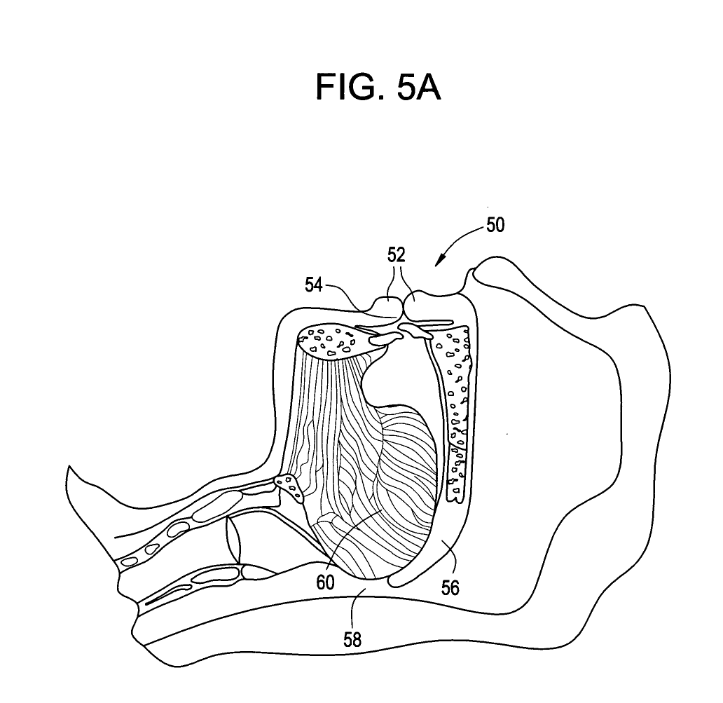

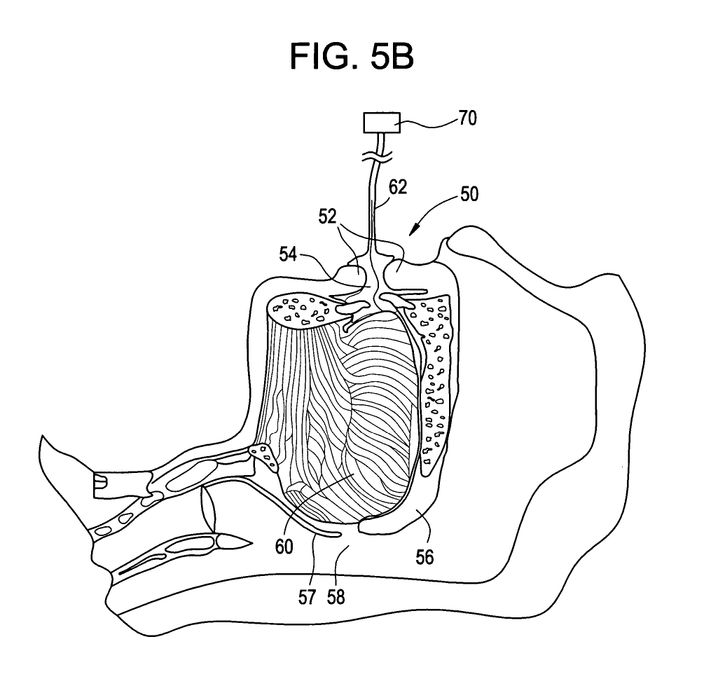

The present invention also provides methods for removing an obstruction in a human airway related to snoring and/or OSA. By way of non-limiting example, FIGS. 5A and 5B illustrate an obstructed airway, and the airway after the obstruction has been removed by a negative pressure generator, respectively. As shown, a sealed cavity 54 is formed within a patient's mouth 50, e.g., using a mouthpiece (not shown) or other suitable device, and the sealed cavity 54 is coupled to a negative pressure generator 70. The negative pressure generator 70 is then activated to withdraw air from the sealed cavity 54, preferably at a continuous rate. In an exemplary embodiment, the negative pressure generator 70 operates at a pressure in the range of about 0 to −100 cm of water, and/or it removes air at a rate of about 1 cc/minute to 50 cc/minute so that the patient does not feel any pressure when the device 70 is operating with an open airway. A person skilled in the art will appreciate that the pressure range of the negative pressure generator may vary over other ranges. For example, it may operate in a pressure range of about −1 to −50 cm of water, and more preferably at a range of about −10 to −40 cm of water. Once the patient is asleep and an obstruction of the airway occurs due, for example, to falling of the patient's tongue 60 and/or soft palate 56 against the posterior pharyngeal wall 58, as shown in FIG. 5A, the blocked airway and the mouthpiece will close the sealed cavity 54 within the patient's mouth. As a result, the negative pressure generator can remove air from the sealed cavity 54 to create a negative pressure that is effective to pull the patient's tongue 60 and/or soft palate 56 up and away from the posterior pharyngeal wall 58, thereby re-opening the airway, as shown in FIG. 5B.

One skilled in the art will appreciate further features and advantages of the invention based on the above-described elements. Accordingly, the invention is not to be limited by what has been particularly shown and described, except as indicated by the appended claims. All publications and references cited herein are expressly incorporated herein by reference in their entirety.

1. A device adapted to remove an obstruction in a human airway, comprising:
a mouthpiece adapted to form a sealed cavity within a human mouth without impinging on a tongue within the mouth; and
a hollow elongate member having a first end coupled to the mouthpiece and in communication with the sealed cavity, and a second end adapted to be coupled to a negative pressure generator that is effective to generate a negative pressure within the sealed cavity in the mouth and/or upper airway in response to a blocked airway.

2. The device of claim 1, wherein the mouthpiece includes upper and lower portions that conform to an anatomy of a human's upper and lower dental structures.

3. The device of claim 2, wherein the upper and lower portions are adapted to maintain upper and lower dental structures at a fixed distance from one another.

4. The device of claim 2, wherein the upper and lower portions are connected to one another by a sidewall extending therebetween.

5. The device of claim 4, wherein the sidewall is adapted to expand the size of the sealed cavity in the mouth.

6. The device of claim 4, wherein the hollow elongate member is coupled to the sidewall.

7. The device of claim 1, wherein the mouthpiece includes a sidewall adapted to be positioned over an opening of a human mouth, and a positioning member adapted to fit within the mouth to maintain the mouthpiece at a fixed position.

8. The device of claim 7, wherein the positioning member includes opposed first and second fixation elements that are adapted to be positioned between upper and lower dental structures.

9. The device of claim 1, further comprising a negative pressure generator coupled to the second end of the hollow elongate member.

10. The device of claim 9, wherein the negative pressure generator comprises a deformable member that is adapted to deform in response to inhalation, and that is biased to an original, un-deformed state such that return to the original, un-deformed state is effective to generate a negative pressure within the sealed cavity when an airway obstruction occurs.

11. The device of claim 10, wherein the deformable member includes at least one one-way valve that is adapted to control air flow into and out of the deformable member.

12. The device of claim 10, wherein the deformable member is adapted to be coupled to a human chest.

13. The device of claim 12, further comprising a mating element adapted to releasably attach the deformable member to the chest.

14. The device of claim 10, wherein the deformable member comprises a deformable ball.

15. The device of claim 2, wherein the lower portion is adapted to be displaced relative to the upper portion.

16. The device of claim 15, wherein the lower portion is adapted to be locked in a desired position in which it is displaced from the upper portion.

17. A method for removing an obstruction in a human airway, comprising:
forming a sealed cavity within a human mouth;
coupling the sealed cavity to a negative pressure generator; and
activating the negative pressure generator to remove air from the sealed cavity at a rate that is effective to create a negative pressure within the sealed cavity when the soft tissues of the upper airway fall against the posterior pharyngeal wall to pull the soft tissues of the upper airway away from the posterior pharyngeal wall, thereby removing the obstruction.

18. The method of claim 17, wherein a mouthpiece is used to form the sealed cavity.

19. The method of claim 18, wherein the mouthpiece is adapted to allow normal swallowing and breathing.

20. The method of claim 18, wherein the mouthpiece does not impinge upon the tongue.

21. The method of claim 18, wherein the mouthpiece includes upper and lower portions that conform to an anatomy of a human's upper and lower dental structures.

22. The method of claim 21, wherein the upper and lower portions are adapted to maintain upper and lower dental structures at a fixed distance from one another.

23. The method of claim 18, wherein the mouthpiece is adapted to expand the size of the sealed cavity in the mouth.

24. The method of claim 18, further comprising a hollow elongate member having a first end coupled to the mouthpiece and in communication with the sealed cavity, and a second end coupled to the negative pressure generator.

25. The method of claim 18, wherein the mouthpiece includes a sidewall adapted to be positioned over an opening of a human mouth, and a positioning member adapted to fit within the mouth to maintain the mouthpiece at a fixed position.

26. The method of claim 17, wherein the negative pressure generator operates at a pressure in the range of about −1 to −100 cm of water.

27. The method of claim 17, wherein in the negative pressure generator removes air from the sealed cavity at a constant rate.

28. The method of claim 27, wherein the rate is in the range of about 1 cc/minute to 50 cc/minute.

29. The method of claim 18, wherein the negative pressure generator is coupled to the human, and inhalation and exhalation by the human is effective to operate the negative pressure generator to allow a negative pressure to be created in the sealed cavity when an airway obstruction occurs.

30. The method of claim 18, wherein the negative pressure generator comprises a deformable member that is adapted to deform in response to inhalation, and that is biased to an original, un-deformed state such return to the original, un-deformed state is effective to generate a negative pressure within the sealed cavity when an airway obstruction occurs.

31. The method of claim 30, wherein the deformable member includes a one-way valve that is effective to release air when the deformable members deforms.

32. The method of claim 30, wherein the deformable member is coupled to a human chest.

33. The method of claim 32, wherein the deformable member includes a mating element adapted to releasably attach the deformable member to the chest.

34. The device of claim 30, wherein the deformable member comprises a deformable ball.
